(19) United States
(12) Patent Application Publication
(10) Pub. No.: US 2020/0030528 A1
(43) Pub. Date: Jan. 30, 2020

(54) Implantable Drug Delivery Device with Infusate Measuring Capabilities

(71) Applicant: Flowonix Medical Incorporated, Mt. Olive, NJ (US)

(72) Inventors: Paul BURKE, Bellingham, MA (US); Steve ADLER, Randolph, NJ (US); Robert Lash, Redwood City, CA (US)

(21) Appl. No.: 16/592,972

(22) Filed: Oct. 4, 2019

Related U.S. Application Data
(63) Continuation-in-part of application No. 15/098,663, filed on Apr. 14, 2016.
(60) Provisional application No. 62/148,457, filed on Apr. 16, 2015.

(51) Int. Cl. A61M 5/145; A61M 5/142; A61M 5/155; A61M 5/168; A61M 5/172

(52) U.S. Cl. CPC A61M 5/14593; H04W 72/04; A61M 5/155; A61M 5/1684; A61M 5/16881; A61M 5/16886; A61M 5/172; A61M 2005/16863; A61M 2205/18; A61M 2205/3306; A61M 2205/3317; A61M 2205/3337; A61M 2205/15; A61M 2205/3375; A61M 2205/3576; A61M 2205/50; A61M 5/14276

(57) ABSTRACT

An implantable drug delivery device and method that includes a sensor device for detecting deflection of a diaphragm at a first time from a first position within a diaphragm chamber of an accumulator of the implantable drug delivery device. A cycle count may be incremented in response to the diaphragm returning to the first position within the diaphragm chamber, each time during cyclical deflections within the diaphragm chamber. A flow rate of infusate may be determined based on a volume of the diaphragm chamber, the cycle count, and an elapsed time since the first time. Once the flow rate is determined, a processor may determine whether the flow rate of infusate is normal based on a comparison of the determined flow rate to at least one threshold flow rate.

### RELATED APPLICATION

This application is a continuation-in-part of U.S. patent application Ser. No. 15/098,663 titled “Implantable Drug Delivery Device with Flow Measuring Capabilities” filed on Apr. 14, 2016 which claims the benefit of priority to U.S. Provisional Application No. 62/148,457, entitled “Implantable Drug Delivery Device with Flow Measuring Capabilities” filed on Apr. 16, 2015, the entire contents of all of which are incorporated herein by reference.

### FIELD

The present invention relates generally to implantable infusion devices for the delivery of medication or other fluids to a patient.

### BACKGROUND

Various implantable devices exist for delivering infusate, such as medication, to a patient. One such device is an implantable valve accumulator pump system. This system includes an electronically controlled metering assembly located between a drug reservoir and an outlet catheter. The metering assembly may include two normally closed solenoid valves that are positioned on the inlet and outlet sides of a fixed volume accumulator. The inlet valve opens to admit a fixed volume of infusate from the reservoir into the accumulator. Then, the inlet valve is closed and the outlet valve is opened to dispense the fixed volume of infusate from the accumulator to an outlet catheter through which the infusate is delivered to the patient. The valves may be controlled electronically via an electronics module, which can optionally be programmed utilizing an external programmer to provide a programmable drug delivery rate. Because the device is typically implanted in the patient's body and not easily accessed while it is operating, it can be difficult to detect when there is a fault condition or other deviation from normal operating conditions of the device.

### SUMMARY

The systems, methods, and devices of the various embodiments provide an implantable drug delivery device that includes a sensor device for detecting deflection of a diaphragm at a first time from a first position within a diaphragm chamber of an accumulator of the implantable drug delivery device. A cycle count may be incremented each time the sensor device detects the diaphragm returns to the first position within the diaphragm chamber during cyclical deflections within the diaphragm chamber. A flow rate of infusate may be determined based on a volume of the diaphragm chamber, the cycle count, and an elapsed time since the first time. Once the flow rate is determined, a processor may determine whether the flow rate of infusate is normal based on a comparison of the determined flow rate to at least one threshold flow rate.

### BRIEF DESCRIPTION OF THE DRAWINGS

The accompanying drawings, which are incorporated herein and constitute part of this specification, illustrate example embodiments of the invention, and together with the general description given above and the detailed description given below, serve to explain the features of the invention.

FIG. 1 is a schematic diagram of an implantable drug delivery system.

FIGS. 2A-2D schematically illustrate a fixed-volume accumulator of a metering assembly and the sequence of steps performed by the metering assembly of the implantable drug delivery system.

FIG. 3 is a schematic diagram of an embodiment implantable drug delivery device that includes a strain gauge sensing device configured to measure a change in position or deflection of a diaphragm of an accumulator.

FIG. 4 is a schematic diagram of an embodiment implantable drug delivery device that includes a capacitive displacement sensor configured to measure a change in position or deflection of a diaphragm of an accumulator.

FIG. 5 is a schematic diagram of an embodiment implantable drug delivery device that includes a light-based sensor configured to measure a change in position or deflection of a diaphragm of an accumulator.

FIG. 6 is a schematic diagram of an embodiment implantable drug delivery device that includes a pressure sensor configured to measure a change in position or deflection of a diaphragm of an accumulator.

FIG. 7 a schematic diagram of an embodiment implantable drug delivery device that includes a sonic-based sensor configured to measure a change in position or deflection of a diaphragm of an accumulator.

FIG. 8 is a process flow diagram illustrating a method of operating an implantable drug delivery device according to an embodiment.

FIG. 9 is a process flow diagram illustrating an embodiment method for monitoring the flow rate of infusate from an implantable drug delivery device according to some embodiments.

FIG. 10 is a process flow diagram illustrating an embodiment method for monitoring the flow rate of infusate from an implantable drug delivery device according to some embodiments.

### DETAILED DESCRIPTION

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the invention or the claims.

The words “exemplary” or “for example” are used herein to mean “serving as an example, instance, or illustration.” Any implementation described herein as “exemplary” or “for example” is not necessarily to be construed as preferred or advantageous over other implementations.

The systems, methods, and devices of various embodiments measure the flow rate of infusate from an implantable drug delivery device by counting accumulator diaphragm displacement cycles of a diaphragm in an accumulator. The various embodiments enable monitoring the flow rate by monitoring cycles of the accumulator diaphragm within the diaphragm chamber and determining a flow rate based on a number of displacement cycles of the diaphragm occurring over a measured or know period of time and the volume displaced by the accumulator in a single displacement cycle. To determine when each cycle starts and stops, various embodiments may detect a change in position (i.e., deflection) of the accumulator diaphragm and determining when the diaphragm returns to that position. In some embodiments, the period of time during which accumulator diaphragm displacement cycles are counted may be determined based on measuring time when cycle counts are started and when cycle counts are stopped. In some embodiments, accumulator diaphragm displacement cycles may be counted for a predetermined amount of time, such as by counting accumulator diaphragm displacement cycles that occur between when a timer is started and the when the timer ends. Various embodiments may include an implantable drug delivery device having a sensor device configured to measure the position or deflection of the diaphragm sufficient to determine when an accumulator diaphragm displacement cycle has completed. The sensor may be an electronically-based sensor, such as strain gauge or capacitive displacement sensor, a light-based sensor, a pressure sensor or a sonically-based sensor.

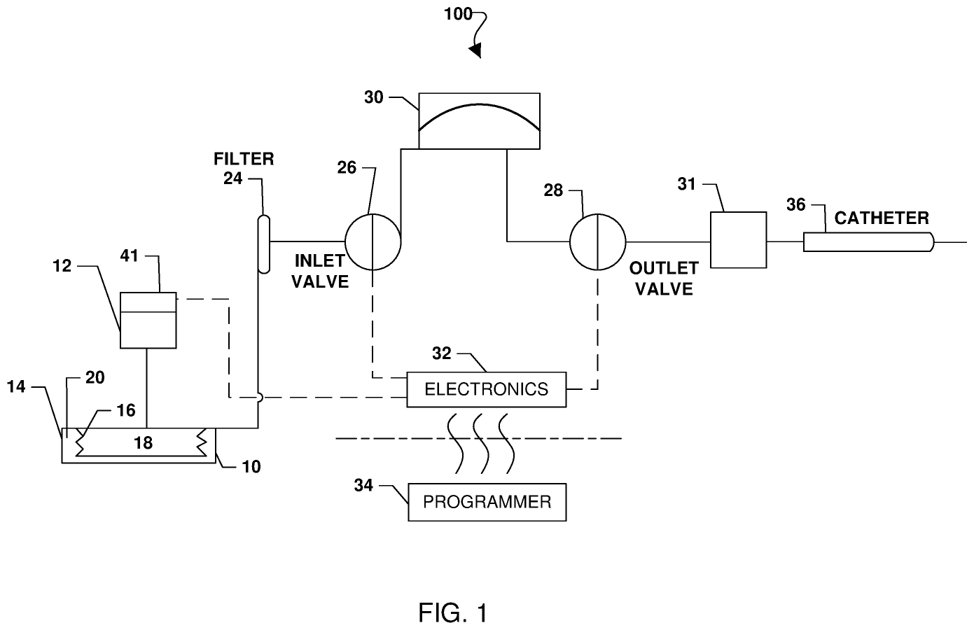

FIG. 1 illustrates an embodiment of an implantable valve accumulator pump system 100 for the delivery of infusate, such as medication. The system 100 may generally include four assemblies. The first major assembly is a rechargeable, constant pressure drug reservoir 10 in series with a bacteria/air filter 24. In one embodiment, the reservoir 10 includes a sealed housing 14 containing a bellows 16. The bellows 16 separates the housing 14 into two parts, a chamber 18 and a second zone 20. The chamber 18 is used to hold the drug or other medicinal fluid. The second zone 20 is normally filled with a two-phase fluid, such as Freon®, that has a significant vapor pressure at body temperature. Thus, as the fluid within the second zone 20 vaporizes, the vapor compresses the bellows 16, thereby pressurizing the drug in the chamber 18. The chamber 18 can be refilled with an infusate via a refill septum 12.

The two-phase fluid helps maintain the chamber 18 under a constant pressure. When the chamber 18 is refilled, the two-phase fluid is pressurized thereby condensing a portion of the vapor within the second zone 20 to the liquid phase. As the chamber 18 is emptied, this liquid vaporizes, thus maintaining the pressure on the bellows 16. Since the infusate in the chamber 18 is under positive pressure, the infusate is urged out of the chamber through a bacterial filter 24 and toward the metering assembly.

The second major assembly is an electronically controlled metering assembly that may include two normally closed solenoid valves 26, 28 that are positioned on the inlet and outlet sides of a fixed volume accumulator 30. The valves are controlled electronically via an electronics module 32, which may be programmed utilizing the external programmer 34. The metering assembly may be designed such that the inlet valve 26 and the outlet valve 28 are never simultaneously open.

The third major assembly is an outlet catheter 36 for medication infusion in a localized area. The delivery of fluid occurs at an infusion site that has a pressure less than the accumulator pressure. This pressure difference forces discharge of the infusate through the catheter 36.

The drug reservoir 10 and electronically controlled metering assembly may be contained within a biocompatible housing, also containing a power source (e.g., battery) that may be implanted within the body of a human or animal patient. The outlet catheter 36 may be integral with the housing, or may be a separate component that is attached to the housing. An access port 31, in communication with the catheter 36, may be provided downstream of the metering assembly. The access port 31 may be used, for example, to manually provide a bolus dose of medication to the patient.

The fourth assembly of the system illustrated in FIG. 1 is an external programmer 34 used to communicate and program the desired medication regimen. In an embodiment, the external programmer 34 may be a handheld unit with a touch screen. The external programmer 34 may provide a wireless data transfer link to a wireless communication transceiver within the implanted electronics module 32 and may be enabled to exchange information with the electronic module 32, including but not limited to battery status, diagnostic information, calibration information, etc. In various embodiments described in further detail below, the electronic module 32 may communicate information regarding the flow rate of infusate from the implantable system 100 to the external programmer 34. In an embodiment, the external programmer 34 may send an instruction to the electronics module 32 to detect the flow rate of infusate from the implantable system according to the embodiments described below. In an embodiment, the electronics module 32 may include a coil configured to send and receive electromagnetic signals to/from the external programmer 34.

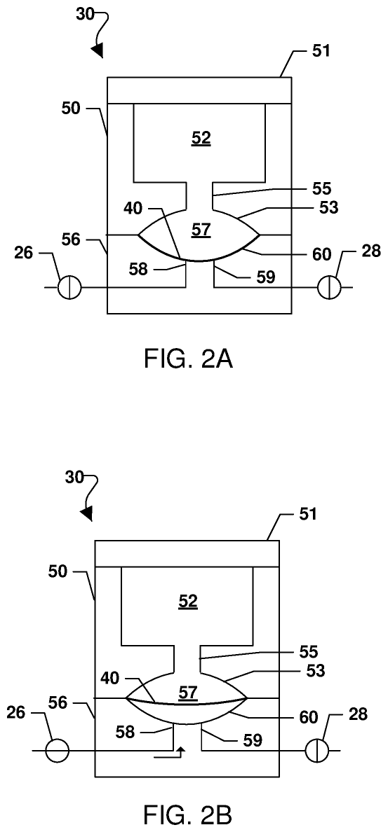

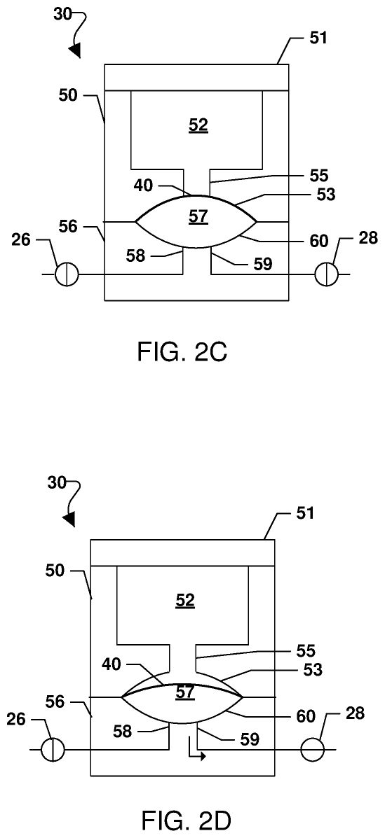

FIGS. 2A-2D schematically illustrate the structure and operation of a fixed volume accumulator 30 of an electronically-controlled metering assembly according to one embodiment. The accumulator 30 may include a housing 50 that together with a cap 51 defines a sealed gas chamber 52. The cap 51 may be secured to the housing 50 using any suitable means, such as laser welding. A suitable gas may be sealed, under positive pressure, within the gas chamber 52. The sealed gas chamber 52 may contain an inert gas such as argon, helium or nitrogen, air, or mixtures of different gases. Alternately, the sealed gas chamber 52 may contain a two-phase fluid. A bottom surface of the housing 50 may define a first (e.g., upper) surface 53 of a diaphragm chamber 57. One or more fluid passages 55 within the housing 50 may connect the gas chamber 52 with the diaphragm chamber 57.

A face plate 56 (which may also be referred to as a spacer plate) may be secured to the bottom surface of the housing 50. An upper surface of the face plate 56 may define a second (e.g., lower) surface 60 of the diaphragm chamber 57. A diaphragm 40 may be located between the housing 50 and the face plate 56 and within the diaphragm chamber 57 defined therebetween. In embodiments, the edges of the diaphragm 40 may be sandwiched between the housing 50 and the face plate 56, and the assembly may be sealed, such as via laser welding. The diaphragm 40 may provide a barrier separating a gas side (e.g., above the diaphragm 40) from a fluid side (e.g., below the diaphragm 40) in the accumulator 30. The face plate 56 may include a fluid inlet port 58 that provides fluid communication between the inlet valve 26 and the diaphragm chamber 57 and a fluid outlet port 59 that provides fluid communication between the outlet valve 28 and the diaphragm chamber 28.

In embodiments, the diaphragm 40 may include a thin, disk-shaped sheet. The diaphragm 40 may include a metal, such as titanium. The diameter and thickness of the diaphragm 40 may be selected to provide a low spring rate over a desired range of deflection. The diaphragm 40 may function as a compliant, flexible wall that separates a fluid (e.g., liquid infusate) from the environment behind it. In the embodiment illustrated in FIGS. 2A-2B, the deflections of the diaphragm 40, illustrated as upward and downward motions, are limited by the first and second surfaces 53, 60 of the diaphragm chamber 57 that act as mechanical stops for the diaphragm 40. In the embodiment illustrated in FIGS. 2A-2B, each of these surfaces 53, 60 are formed having a shallow concave profile that acts as a contour stop for the diaphragm 40. The dimensions of the contour may be chosen to match the general profile of the diaphragm 40 when it is deflected or biased by a predetermined fixed volume. This predetermined fixed volume corresponds to the volume that is metered by the accumulator 30. In other embodiments, one of the surfaces 53, 60 may have a generally flat profile that corresponds to the profile of the diaphragm in a flat, undeflected state, while the other surface may correspond to the profile of the diaphragm in a deflected state.

In some embodiments, the second (e.g., lower) surface 60 of the diaphragm chamber 57 may include one or more channels formed in the surface 60 to maximize wash out of fluid and minimize dead volume within the chamber 57. For example, the surface 60 may be formed with an annular groove intersected by a trough connecting the inlet and outlet ports 58, 59, such as described in U.S. Pat. No. 8,273,058 to Burke et al., which is incorporated herein by reference for details of the diaphragm chamber.

FIG. 2A illustrates the accumulator 30 in a state in which both the inlet valve 26 and the outlet valve 28 are closed, and the diaphragm 40 deflects downward (in the orientation presented in FIG. 2A) as a result of the bias from the gas pressure in the gas chamber 52 and in the gas side of the diaphragm chamber 57. In this portion of the pumping cycle, there is no liquid infusate in the diaphragm chamber 57.

FIG. 2B shows the accumulator 30 after the inlet valve 26 is opened, while the outlet valve 28 remains closed. The pressure of the liquid infusate from reservoir 10 (see FIG. 1) is sufficient to overcome the bias of the pressurized gas against the back side of the diaphragm 40, causing the diaphragm 40 to separate from the second (lower) surface 60 of the diaphragm chamber 57. The infusate begins to flow into the diaphragm chamber 57 through the inlet port 58, as indicated by the arrow in FIG. 2B. As the infusate fills the diaphragm chamber 57, the bias from the fluid pressure in the chamber 57 causes the diaphragm 40 to deflect upwards (in the orientation presented in FIG. 2B) towards the first (upper) surface 53 of the diaphragm chamber 57.

FIG. 2C shows the accumulator 30 filled with infusate to its fixed or desired volume. The diaphragm 40 is biased against the first (upper) surface 53 of the diaphragm chamber 57, which acts as a mechanical stop for the diaphragm 40. When the accumulator 30 is filled with infusate, the inlet valve 26 is closed, as shown in FIG. 2C.

FIG. 2D shows the accumulator 30 after the outlet valve 28 is opened while the inlet valve 26 remains closed. The infusate begins to flow out of the diaphragm chamber 57 through the outlet port 59 and the catheter 36 (see FIG. 1), as indicated by the arrow in FIG. 2D. As the infusate empties the accumulator, the diaphragm 40 separates from the first (upper) surface 53 of the diaphragm chamber 57. The bias from the gas pressure in the gas chamber 52 and in the gas side of the diaphragm chamber 57 causes the diaphragm 40 to deflect downwards (in the orientation presented in FIG. 2D) towards the second (lower) surface 60 of the diaphragm chamber 57. When the chamber 57 is completely emptied of infusate, the diaphragm 40 is biased against the second (lower) surface 60 of the diaphragm chamber 57, which acts as a mechanical stop for the diaphragm 40. The outlet valve 28 is then closed and the accumulator 30 is again in the state shown in FIG. 2A. The pumping cycle illustrated in FIGS. 2A-2D may then be repeated. The accumulator 30 thus stores and discharges predetermined volume spikes of infusate at a frequency defined by the cycling rate of the inlet and outlet valves 26, 28 of the accumulator 30. The nominal flow rate of infusate from the system 100 may be controlled by controlling the cycling rate of the inlet and outlet valves 26, 28 of the accumulator 30.

In operation, the programmed flow rate of infusate from the system may not represent the actual rate of infusate being delivered to the patient for a variety of reasons. For example, there may be a blockage or occlusion of the infusate flow in the catheter or elsewhere in the device, a malfunctioning valve, a leak in the device, or another fault condition. Any one or combination of these conditions may result in a situation in which more or less than the desired amount of the infusate is being delivered to the patient in a given time period. This can result in reduced efficacy of the treatment regimen and can potentially be dangerous to the patient. Further, it has generally not been possible to directly measure the amount of infusate being delivered to the patient from the catheter (e.g., using a conventional fluid flow meter) since the infusate is typically delivered to a confined and sensitive area inside the patient's body where the use of conventional flow meters is impractical.

The various embodiments include methods and systems for indirectly measuring the flow rate of an implantable drug delivery device by measuring the movement of a diaphragm in a fixed-volume accumulator. Embodiments include various systems and methods for measuring a change in position or deflection of the diaphragm over time to determine the rate of flow of infusate from the accumulator. For example, referring to the fixed volume accumulator 30 illustrated in FIGS. 2A-2D, the amount of time it takes for the diaphragm 40 to move from the position shown in FIG. 2C (i.e., with the diaphragm biased against the first (upper) surface 53 of the diaphragm chamber 57) to the position shown in FIG. 2A (e.g., with the diaphragm biased against the second (lower) surface 60 of the diaphragm chamber 57) is directly related to the flow rate of the known volume of infusate that is dispensed from the accumulator during a pumping cycle. This time may vary based on the amount of flow restriction in the catheter or elsewhere in the system. In some cases, such as when there is a blockage or leak in the flow path of the device, the diaphragm chamber 57 may not completely fill or discharge during each pumping cycle (e.g., such that the diaphragm does not fully deflect to the positions illustrated in FIG. 2A and/or 2C during the pumping cycle). This may be detected by measuring the change in position or deflection of the diaphragm as a function of time.

Various embodiments may include an implantable drug delivery device that includes a diaphragm sensor for detecting a change in position or deflection of a diaphragm of a fixed volume accumulator. An electronics module connected to the diaphragm sensor may monitor the detected change in position or deflection of the diaphragm as a function of time to determine whether the flow rate of the device satisfies at least one pre-determined criteria. The electronics module may be configured such that in response to determining that the flow rate does not satisfy the pre-determined criteria, the electronics module may take an appropriate action, such as sending a wireless signal providing a notification to a user of the device and/or medical personnel, adjusting the cycling rate of the fixed-volume accumulator to bring the flow rate within the pre-determined criteria, and/or shutting down the device to prevent further infusion of the medication.

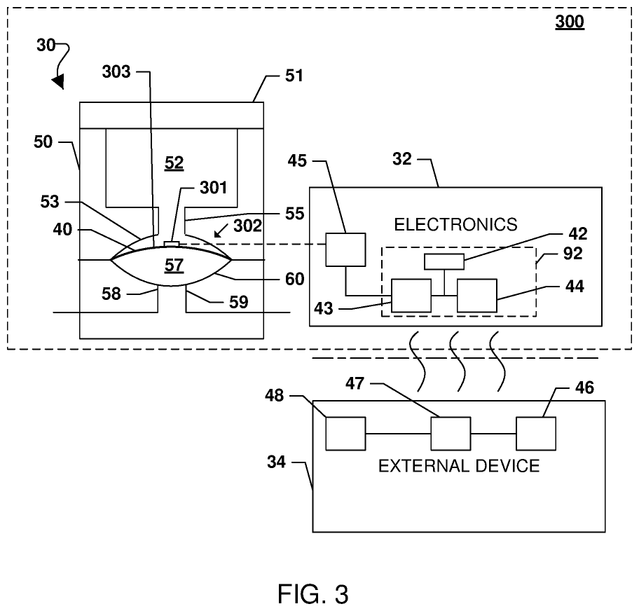

The diaphragm sensor may be any suitable diaphragm sensor that is configured to detect a change in position or deflection of the diaphragm 40. FIG. 3 illustrates a first embodiment of an implantable drug delivery device 300 that includes an electronically-based diaphragm sensor 302 configured to measure a change in position or deflection of a diaphragm 40 of an accumulator 30 as a function of time. In this embodiment, the electronically-based diaphragm sensor 302 may include at least one strain gauge 301. The at least one strain gauge 301 may be located on a surface 303 of the diaphragm 40 that is exposed to the gas from the sealed gas chamber 52 and opposite the surface of the diaphragm 40 that is exposed to the infusate (the surface 303 may alternately be referred to as the “back side” of the diaphragm 40). Alternatively, or in addition, one or more strain gauges may be located on the “front side” of the diaphragm (i.e., the surface that is exposed to the infusate in the diaphragm chamber 57).

The at least one strain gauge 301 may include any suitable type of diaphragm sensor device for converting mechanical strain to a proportional electrical signal. For example, the at least one strain gauge 301 may include a bonded foil strain gauge, a bonded semiconductor strain gauge (e.g., a piezoresistor), a thin film strain gauge (e.g., a strain gauge formed by vapor deposition or sputtering of an insulator and gauge material onto the surface of the diaphragm), and/or a diffused or implanted semiconductor strain gauge. The at least one strain gauge may be calibrated to measure the strain corresponding to the displacement (i.e. deflection) of the diaphragm 40 between a flat, resting-state position to the maximum upward and/or downward deflection positions of the diaphragm 40 within the accumulator 30 (i.e., the positions of the diaphragm shown in FIGS. 2A and 2C).

In the device 300 illustrated in FIG. 3, the electronics module 32 may include a controller 92. In an embodiment, the controller 92 may include a processor 43 coupled to a memory 44. The processor 43 may be any type of programmable processor, such as a microprocessor or microcontroller, which may be configured with processor-executable instructions to perform the operations of the embodiments described herein. Processor-executable software instructions may be stored in the memory 44 from which they may be accessed and loaded into the processor 43. The processor 43 may include internal memory sufficient to store the application software. The memory 44 may be volatile, nonvolatile such as flash memory, or a mixture of both.

In an embodiment, the controller 92 may be coupled to a strain gauge monitoring circuit 45 of the diaphragm sensor 302. The strain gauge monitoring circuit 45 may measure a change in an electrical characteristic (e.g., resistance) of the at least one strain gauge 301 corresponding to the strain experienced by the strain gauge 301. The strain gauge monitoring circuit 45 may include a four-gauge Wheatstone bridge circuit, for example. The electronics module 32 may also include a clock generator that generates timing signals so that each of the measured strain values may be associated with a particular measurement time. The controller 92 may compare the measured strain from the monitoring circuit 45 to pre-determined strain values corresponding to different deflection positions of the diaphragm 40 within the accumulator 30. The pre-determined strain values may be stored in the memory 44, such as in the form of a look-up table, for example. The controller 92 may use the measured strain values from the monitoring circuit 45 and the known pre-determined values corresponding to different deflection positions of the diaphragm 40 to determine the change in position or deflection of the diaphragm 40 (i.e., the amount of upward and/or downward deflection of the diaphragm 40 as oriented in the figures) as a function of time. As discussed above, the change in position or deflection of the diaphragm as a function of time may be directly related to the rate at which the infusate is pumped from the accumulator. The controller 92 may be configured to determine whether the detected change in position or deflection of the diaphragm as a function of time is within normal operating parameters (i.e., the detected change of position or deflection of the diaphragm as a function of time corresponds to a clinically acceptable flow rate of the infusate). In some embodiments, the controller 92 may not translate the measured strain values into deflection values, and instead may be configured to determine whether the detected change in measured strain values over a period of time is within normal operating parameters (i.e., the detected change in measured strain values over time corresponds to a clinically acceptable flow rate of the infusate).

The controller 92 may be configured to provide a notification to the user, such as by sending a message to an external device 34, when the detected motion of the diaphragm is determined to be outside normal operating parameters (i.e., not within such parameters). The external device 34 may be a programmer as described above, or alternately another external device may be configured to communicate with the implantable device 300 via a wireless data transfer link.

In various embodiments, the external device 34 may include a processor 47 coupled to a memory 46 and to an indicator 48. Software instructions may be stored in the memory 46 before they are accessed and loaded into the processor 47. The processor 47 may be configured to activate the indicator 48 to provide a notification (e.g., an alarm) to the user when the external device 34 receives a message from the controller 92 of the implantable device 300 indicating that the detected motion of the diaphragm and/or the flow rate of infusate is not within pre-determined parameters. The indicator 48 may be a display, a speaker for an audio or sound message, and/or a vibrator to generate haptic feedback, for example. The processor 47 of the external device 34 may also be configured to notify medical personnel who may be located remotely, such as via a wireless communication network, in response to receiving messages from the controller 92 of the implantable device 300.

In some embodiments, the controller 92 of the implantable device 300 may be configured to detect the motion of the diaphragm on a pre-determined and/or periodic basis (e.g., every hour, every 12 hours, etc.). The scheduled times and/or frequency in which the controller 92 detects the motion of the diaphragm may be varied based on instructions received from the external device 34. Alternatively or in addition, the controller 92 of the implantable device 300 may detect the motion of the diaphragm “on demand” in response to a request or command from the external device 34. In some embodiments, the controller 92 of the implantable device 300 may be configured to detect the motion of the diaphragm 40 continuously or frequently over the duration of a treatment regimen.

In some embodiments, the controller 92 of the implantable device 300 may forward a plurality of raw measurements from the strain gauge monitoring circuit 45 to the external device 34. The processor 47 of the external device 34 may use the raw measurement values to determine the change in diaphragm position or deflection over time and/or the flow rate of infusate from the device 300. The processor 47 of the external device 34 may compare the calculated value(s) to one or more stored threshold values to determine whether the flow rate is within clinically acceptable parameters. In other embodiments, the controller 92 of the implantable device 300 may determine an infusate flow rate value based on the detected change in diaphragm position or deflection over time, and may forward the determined infusate flow rate to the external device 34. The external device 34 may display the flow rate value on the indicator 48.

In some embodiments, the diaphragm sensor 302 may be used to count diaphragm displacement cycles (equivalent to pumping cycles) of the accumulator, such as those illustrated in FIGS. 2A-2D. The controller 92 may be configured to detect each accumulator diaphragm displacement cycle from a reference position of the diaphragm 40. For example, the reference position may be that shown in either FIG. 2A, which corresponds to the diaphragm 40 fully deflected downward and the diaphragm chamber 57 fully discharged or FIG. 2C with the diaphragm 40 fully deflected upward and the diaphragm chamber 57 fully filled, although the reference position of the diaphragm for determining a displacement cycle may be any position between fully deflected upward and fully deflected downward. If the reference position is either of the fully deflected positions, the processor 43 may register or count one diaphragm displacement cycle when the diaphragm 40 returns to the reference position. If the reference position is somewhere between the fully deflected upward and downward positions, such as those shown in either FIG. 2B or 2D, the processor 43 may register or count one diaphragm displacement cycle every other time the diaphragm 40 passes the reference position (or count every passing and divide by two) because the diaphragm passing the non-fully deflected reference position twice during one complete displacement cycle; once as the diaphragm 40 moves toward the fully deflected upward position and then again as the diaphragm 40 moves toward the fully deflected downward position.

In some embodiments, the processor 43 of the implantable device 300 may count each accumulator diaphragm displacement cycle using a counting circuit 42. The counting circuit 42 may have its own memory or reserve a portion of the memory 44 of the controller 92 and/or the memory 46 of the external device 34 to store a value associated with the diaphragm displacement cycle count. In some embodiments, the processor 43 may store an accumulator diaphragm displacement cycle count for the life of the accumulator 30. For example, at the time of manufacture, the accumulator diaphragm displacement cycle count may be initialized to zero, and would represent the total number of actual accumulator diaphragm displacement cycles performed by the pump to date. This is similar, but distinct from the valve count, which tracks the cycle of the valves (e.g., 26, 28). Every time the processor 43 senses the completion of an accumulator diaphragm displacement cycle, the processor 43 may increment the memory location by 1.

In some embodiments, the electronics module 32 may also include a clock that is capable of determining a duration (e.g., in seconds, minutes, etc.) between two events, particularly between different accumulator diaphragm displacement cycles. Such a clock may be a real-time clock set (at least initially) to a real world time, an arbitrary clock that measures the passage of time but is not linked or correlated to a real world time, or a count-down or count-up timer that expires after a predetermined interval. Using any of such clocks, the processor 43 may determine the interval of time during which accumulator diaphragm displacement cycles were counted, and calculate the infusate flow rate based on the volume displaced by the diaphragm (which may be a constant stored in memory) times the count of accumulator diaphragm displacement cycles divided by the time during which the counts were made.

The flow rate calculated from the accumulator diaphragm displacement cycle counts may be used to detect irregularities in the infusate flow rate. For example, a high flow rate threshold (e.g., 10-15% above a prescribed flow rate) may be set to determine when there is an excessive flow, which may trigger a cut off of the implantable drug delivery device 100. Similarly, a low flow rate threshold (e.g., 10-15% below a prescribed flow rate) may be set to determine when there is too low a flow rate. A low flow rate may be caused by a partial or complete obstruction inside the accumulator due to precipitates, catheter kinking, drug incompatibility with valve seat material, or other reasons.

In response to detecting that the flow rate has reached or exceeded the high flow rate threshold or has reached or dropped below the low flow rate threshold, an alarm and/or message may be generated. An alarm may be an audible or haptic alert to notify the patient or caregiver that the pump has either stopped working or is providing a significantly reduced infusate delivery. The message may notify the patient, care givers, family members, physicians or other medical providers. This may give physicians an opportunity to prescribe or provide supplemental medication to prevent withdrawal, even perhaps through alternate routes of administration such as oral, I.V., etc.).

By determining the infusate flow rate by counting accumulator diaphragm displacement cycles, a less expensive diaphragm sensor may be used, since a precise determination of the position of the diaphragm is not required. Similarly, counting accumulator diaphragm displacement cycles may eliminate the need for calibrating the diaphragm sensor. Thus, various embodiments may enable a less expensive but more reliable and easier to use implantable infusate pump that is capable of determining its actual infusate flow rate.

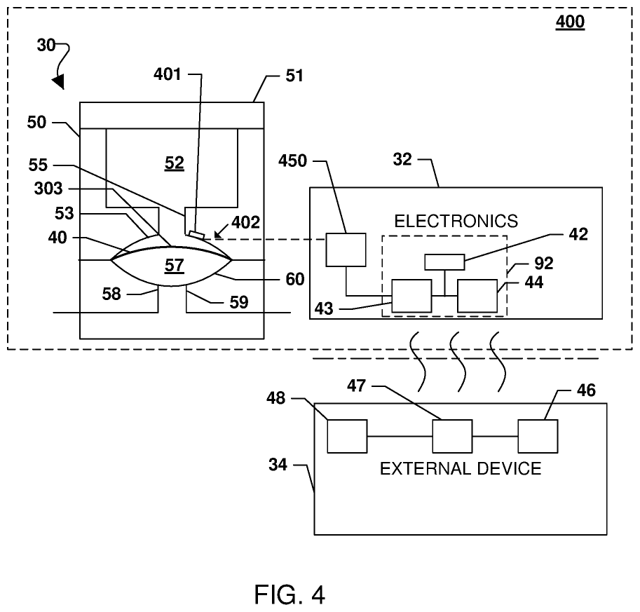

FIG. 4 illustrates a second embodiment of an implantable drug delivery device 400 that includes an electronically-based diaphragm sensor 402 configured to measure a change in position or deflection of a diaphragm 40 of an accumulator 30 as a function of time. In this embodiment, the electronically-based diaphragm sensor 402 may include at least one capacitive displacement sensor 401. Capacitive displacement sensors are noncontact devices that are configured to measure the capacitance between a probe 401 (e.g., an electrode surface) and a target conductive surface (e.g., the surface 303 of the diaphragm 40). The areas of the probe 401 and target surface 303 and the dielectric constant of the material (e.g., gas) between the probe 401 and target surface 303 may be considered constant, in which case the capacitance between the probe 401 and the target surface 303 is proportionally related to the distance between the probe 401 and the target surface 303. Due to this proportional relationship, the diaphragm sensor 402 may measure changes in capacitance as the target surface 303 moves with respect to the probe 402, and a processor may use the measured changes to calculate distance measurements, such as a relative change in the separation distance.

In the embodiment illustrated in FIG. 4, the probe 401 is located proximate to the first (upper) surface 53 of the diaphragm chamber 57, and is configured to measure the displacement of the diaphragm 40 from the first (upper) surface 53 of the chamber 57. Alternatively or in addition, at least one probe 401 may be located proximate to the second (lower) surface 60 of the diaphragm chamber 57 and may be configured to measure the displacement of the diaphragm 40 from the second (lower) surface 60. In other embodiments, a probe 401 may be located on the diaphragm 40 configured to measure the distance between the diaphragm 40 and at least one surface 53, 60 of the diaphragm chamber 57 as the diaphragm moves (i.e., deflects).

The implantable drug delivery device 400 of the embodiment illustrated in FIG. 4 may be similar to the device 300 described above with reference to FIG. 3, and may include an electronics module 32 having a controller 92 comprising a processor 43 and memory 44 as described above. The controller 92 may be coupled to a capacitance monitoring circuit 450 connected to the probe 401 and configured to measure the capacitance between the probe 401 and the surface 303 of the diaphragm 40 as the diaphragm 40 moves within the chamber 57. The controller 92 may be configured to determine changes in the position or deflection of the diaphragm 40 over time based on changes in the measured capacitance. As discussed above, the change in position or deflection of the diaphragm as a function of time may be directly related to the rate at which the infusate is pumped from the accumulator. The controller 92 may be configured to determine whether the detected change in position or deflection of the diaphragm over a period of time is within normal operating parameters (i.e., the detected change of position or deflection of the diaphragm as a function of time corresponds to a clinically acceptable flow rate of the infusate). In some embodiments, the controller 92 may not translate capacitance measurements into distance values, and instead may be configured to determine whether the detected change in capacitance over a period of time is within normal operating parameters (i.e., the detected change in capacitance over time corresponds to a clinically acceptable flow rate of the infusate).

When the detected motion of the diaphragm (or changes in capacitance) is determined to be not within normal operating parameters, the controller 92 may be configured to provide a notification to the user, such as by sending a message to an external device 34. The operation of the device 400 of the embodiment illustrated in FIG. 4 may be substantially similar to the device 300 as described above.

In addition to a mechanical strain gauge and/or capacitive displacement diaphragm sensor as described above, other electronically-based diaphragm sensors may be used to detect the change in position or deflection of the diaphragm 40 as a function of time. For example, the electronically-based diaphragm sensor according to various embodiments may include an eddy current diaphragm sensor and/or an inductive displacement diaphragm sensor.

In various embodiments, the diaphragm sensor 402 may be used, similar to the diaphragm sensor 302 described above, to count accumulator diaphragm displacement cycles, and thus pumping cycles of the accumulator. The controller 92 may be configured to detect each accumulator diaphragm displacement cycle from a reference position of the diaphragm 40. Similarly, the processor 43 of the implantable device 400 may register count each accumulator diaphragm displacement cycle using a counting circuit 42.

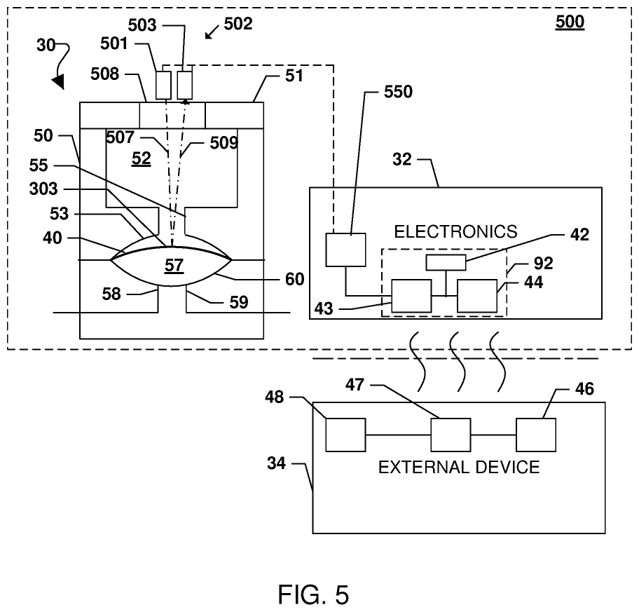

FIG. 5 illustrates a third embodiment of an implantable drug delivery device 500 that includes a light-based diaphragm sensor 502 configured to measure a change in position or deflection of a diaphragm 40 of an accumulator 30 as a function of time. Various devices are known for measuring distance using light signals. A light-based distance measuring device may include a light source 501 (e.g., a laser, LED, etc.) that transmits a beam 507 of radiation (e.g., visible light, UV and/or IR radiation) that is reflected off of a target. The reflected beam 509 is received by a light diaphragm sensor 503 (e.g., a photodiode sensor, a charged coupled device (CCD) sensor, a CMOS-based light sensor, etc.). The distance to the reflective target may be determined using one or more known techniques, such as triangulation, time-of-flight, phase shift, interferometry, chromatic confocal methods, etc. In the embodiment illustrated in FIG. 5, the light beam is reflected off a surface 303 of the diaphragm 40 as the diaphragm 40 deflects within the accumulator 30, and the light-based diaphragm sensor 502 detects the change in position or deflection of the diaphragm 40 over time.

In the embodiment illustrated in FIG. 5, the light source 501 may be located outside of the housing 50 of the accumulator 30 and direct the beam 507 through a transparent window 508 provided in the cap 51 of the housing 50. The beam 507 may be directed through the sealed gas chamber 52 and passage 55 into the diaphragm chamber 57, where the beam 507 is reflected off of the surface 303 of the diaphragm 40. The diaphragm 40 may have a mirror surface 303 to enhance the reflection of the beam. The reflected beam 509 may travel through the passage 55, gas chamber 52 and window 508 and be detected by a light sensor 503 that is located outside of the housing 50 of the accumulator 30. Various other configurations for a light-based diaphragm sensor for measuring displacement of a diaphragm in a fixed-volume accumulator may be used. For example, the light source 501 and/or light sensor 503 may be located within the housing 50, such as within the sealed gas chamber 52, or may be located within the diaphragm chamber 57 (e.g., within surfaces 53 or 60).

The embodiment implantable drug delivery device 500 shown in FIG. 5 may be similar to the devices 300 and 400 described above, and may include an electronics module 32 having a controller 92 comprising a processor 43 and memory 44, as described above. The electronics module 32 may also include a light sensor control circuit 550 coupled to the light source 501 and the light sensor 503 for controlling the operation of the source 501 and light sensor 503 and for generating an electronic signal representation of the reflected light radiation received at the light sensor 503. The controller 92 may be coupled to the light sensor control circuit 550 and may determine changes in the position or deflection of the diaphragm 40 over time based on the electronic signal representation of the reflected light radiation received at the light sensor 503. The controller 92 may use any of the methods described above, including without limitation triangulation, time-of-flight, phase shift, interferometry, and chromatic confocal techniques, to determine the change in position or deflection of the diaphragm 40 over time. As discussed above, the change in position or deflection of the diaphragm as a function of time may be directly related to the rate at which the infusate is pumped from the accumulator. The controller 92 may be configured to determine whether the detected change in position or deflection of the diaphragm as a function of time is within normal operating parameters (i.e., the detected change of position or deflection of the diaphragm as a function of time corresponds to a clinically acceptable flow rate of the infusate). In some embodiments, the controller 92 may not translate measurements from the light sensor into distance values, and instead may be configured to determine whether the detected changes in measured light characteristics (e.g., time of flight, phase shift, interference, etc.) over a period of time are within normal operating parameters (i.e., the detected changes in measured light characteristics over time correspond to a clinically acceptable flow rate of the infusate).

When the detected motion of the diaphragm is determined to be not within normal operating parameters, the controller 92 may be configured to provide a notification to the user, such as by sending a message to an external device 34. The operation of the device 500 may be substantially similar to the operation of the devices 300 and 400 as described above.

In various embodiments, the diaphragm sensor 502 may be used, similar to the diaphragm sensors 302, 402 described above, to count accumulator diaphragm displacement cycles, and thus pumping cycles of the accumulator.

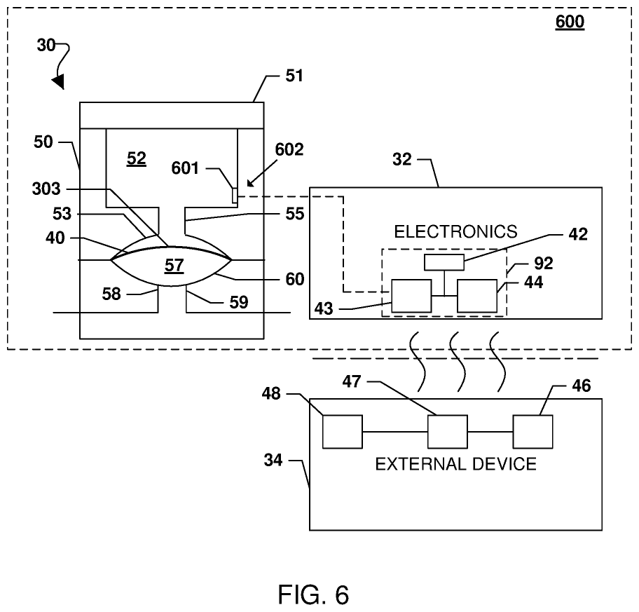

FIG. 6 illustrates a fourth embodiment of an implantable drug delivery device 600 that includes a pressure sensor 602 configured to measure a change in pressure that is related to a change in position or deflection of a diaphragm 40 of an accumulator 30 as a function of time. The pressure sensor 602 may include a pressure transducer 601 that may be located within or in fluid communication with the sealed gas chamber 52 of the accumulator 30. The pressure transducer 602 may be calibrated to detect small changes in the fluid pressure within the chamber 52 as the diaphragm 40 deflects within the diaphragm chamber 57 and may output an electronic signal representing the detected pressure.

The embodiment implantable drug delivery device 600 shown in FIG. 6 may be similar to the devices 300, 400 and 500 described above, and may include an electronics module 32 having a controller 92 comprising a processor 43 and memory 44, as described above. The controller 92 may be coupled to the pressure sensor 602, and may be configured to compare the pressures measured by the pressure sensor 602 to pre-determined pressure values corresponding to different deflection positions of the diaphragm 40 within the accumulator 30. The pre-determined pressure values may be stored in the memory 44 in the form of a look-up table, for example. The controller 92 may use the measured pressure values and the known pre-determined pressure values corresponding to different deflection positions of the diaphragm 40 to determine the change in position or deflection of the diaphragm 40 (i.e., the amount of upward and/or downward deflection of the diaphragm 40) as a function of time. As discussed above, the change in position or deflection of the diaphragm as a function of time may be directly related to the rate at which the infusate is pumped from the accumulator. The controller 92 may be configured to determine whether the detected change in position or deflection of the diaphragm as a function of time is within normal operating parameters (i.e., the detected change of position or deflection of the diaphragm as a function of time corresponds to a clinically acceptable flow rate of the infusate). In some embodiments, the controller 92 may not translate pressure measurements into distance or deflection values, and instead may be configured to determine whether the detected change in pressure over a period of time is within normal operating parameters (i.e., the detected change in pressure over time corresponds to a clinically acceptable flow rate of the infusate).

When the detected motion of the diaphragm is determined to be not within normal operating parameters, the controller 92 may be configured to provide a notification to the user, such as by sending a message to an external device 34. The operation of the device 600 may be substantially similar to the operation of the devices 300, 400 and 500 as described above.

In various embodiments, the pressure sensor 602 may be used, similar to the diaphragm sensors 302, 402, 502 described above, to count accumulator diaphragm displacement cycles, and thus pumping cycles of the accumulator. The controller 92 may be configured to detect each accumulator diaphragm displacement cycle from a reference position of the diaphragm 40. The processor 43 of the implantable device 600 may count each accumulator diaphragm displacement cycle using the counting circuit 42.

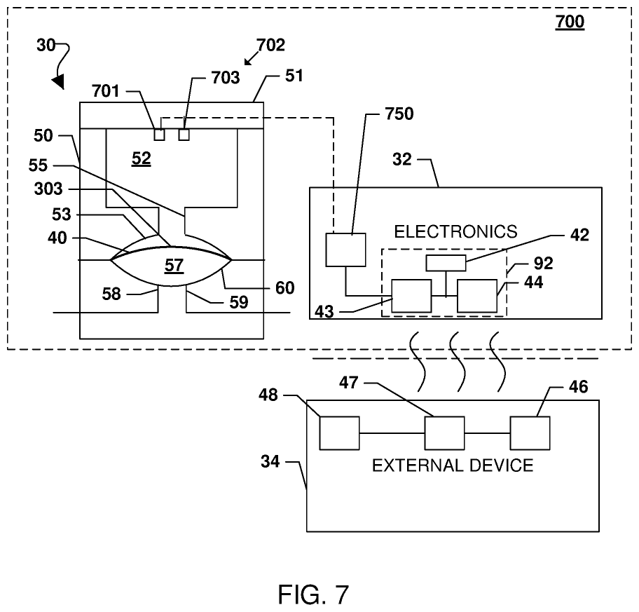

FIG. 7 illustrates a fifth embodiment of an implantable drug delivery device 700 that includes a sonically-based diaphragm sensor 702 configured to measure a change in position or deflection of a diaphragm 40 of an accumulator 30 as a function of time. Various techniques may be used for measuring the displacement of the diaphragm 40 using sonic signals. For example, a source 701 of sonic energy (e.g., a sonic transducer) may generate an acoustic signal (e.g., within an audible, ultrasonic or infrasonic range) within the sealed gas chamber 52 as shown in FIG. 7, or alternatively within the diaphragm chamber 57 (either above or below the diaphragm 40). As the diaphragm deflects within the diaphragm chamber 57, the fluid volume both above and below the diaphragm varies. This variation in volume may change one or more characteristics of the acoustic signal, such a harmonic frequency of the signal, in a manner that may be detected by a sonic sensing device 703. The source 701 of sonic energy and the sonic sensing device 703 are shown as separate devices in FIG. 7, although it will be understood that a single component (e.g., a transducer) may be used to both transmit a sonic energy pulse and receive a reflected pulse (e.g., echo).

The embodiment implantable drug delivery device 700 shown in FIG. 7 may be similar to the devices 300, 400, 500 and 600 described above, and may include an electronics module 32 having a controller 92 including a processor 43 and memory 44, as described above. The electronics module 32 may also include a sonic sensor control circuit 750 coupled to the sonic source 701 and sensing device 703 for controlling the operation of the source 701 and the sensing device 703 and for generating an electronic signal representation of the sonic signal received at the sensing device 703. The controller 92 may be coupled to the sonic sensor control circuit 750 and may determine changes in the position or deflection of the diaphragm 40 over time based on the electronic signal representation of the sonic signal received at the sensing device 703. As discussed above, the change in position or deflection of the diaphragm as a function of time may be directly related to the rate at which the infusate is pumped from the accumulator. The controller 92 may be configured to determine whether the detected change in position or deflection of the diaphragm as a function of time is within normal operating parameters (i.e., the detected change of position or deflection of the diaphragm as a function of time corresponds to a clinically acceptable flow rate of the infusate). In some embodiments, the controller 92 may not translate changes in the received sonic signal into distance values, and instead may be configured to determine whether the detected changes in received sonic signals over a period of time is within normal operating parameters (i.e., the detected changes in sonic signals over time correspond to a clinically acceptable flow rate of the infusate).

When the detected motion of the diaphragm is determined to be not within normal operating parameters, the controller 92 may be configured to provide a notification to the user, such as by sending a message to an external device 34. The operation of the device 700 may be substantially similar to the operation of the devices 300, 400, 500 and 600 as described above.

Various sonically-based diaphragm sensors may be used to detect the change in position or deflection of the diaphragm 40 as a function of time. For example, a sonically-based diaphragm sensor according to various embodiments may use a Doppler, pulse echo and/or sonar technique to measure the displacement of the diaphragm 40 over time.

In various embodiments, the diaphragm sensor 702 may be used, similar to the diaphragm sensors 302, 402, 502 and the pressure sensor 602 described above, to count accumulator diaphragm displacement cycles, and thus pumping cycles of the accumulator. The controller 92 may be configured to detect each accumulator diaphragm displacement cycle from a reference position of the diaphragm 40. The processor 43 of the implantable drug delivery device 700 may count each accumulator diaphragm displacement cycle using the counting circuit 42.

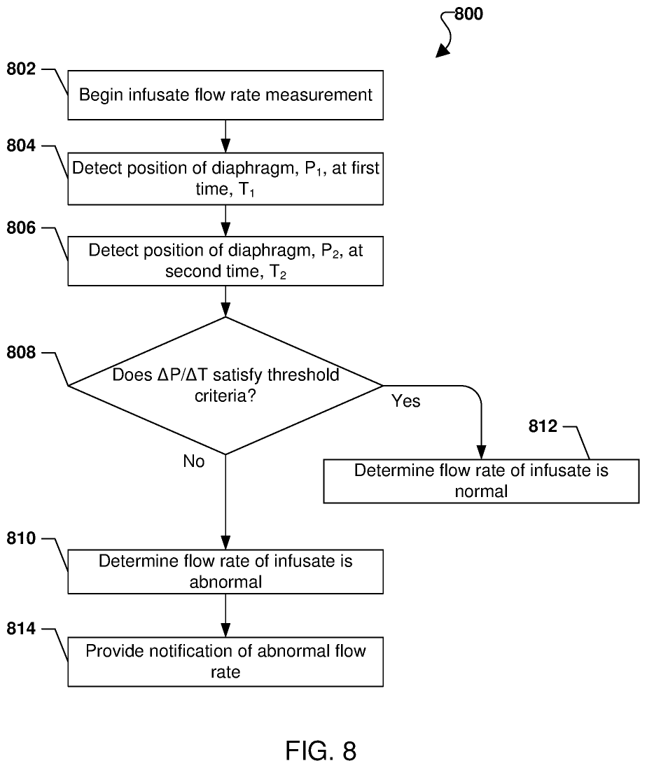

FIG. 8 illustrates an embodiment method 800 for monitoring the flow rate of infusate from an implantable drug delivery device by measuring the movement of a diaphragm in an accumulator of the implantable drug delivery device. An electronics module 32 such as described above may detect the displacement (i.e., the amount of deflection) of the diaphragm as a function of time.

In block 802, the electronics module 32 may begin the flow rate measurement. In an embodiment, the electronics module 32 may begin the flow rate measurement at a pre-determined time or may begin the measurement in response to a command that is received from an external device 34, such as an external programmer.

In block 804, the electronics module 32 may detect the position or deflection of the diaphragm, P₁, at a first time, T₁. For example, the electronics module 32 may detect the position (i.e., the deflection) of the diaphragm when the accumulator 30 is in a filled state, such as shown in FIG. 2C, where the diaphragm 40 is in a maximum (e.g., upwardly) deflected position. The initial time, T₁, may correspond to the time at which the outlet valve 28 of the accumulator 30 is opened and the infusate begins to empty from the accumulator (see FIG. 2D). Thus, in some embodiments the electronics module 32 may synchronize the detection of the diaphragm position P₁ with the opening of outlet valve 28. Alternately, in some embodiments the electronics module 32 may detect the position P₁ of the diaphragm 40 at any arbitrary time during the fill/empty cycle of the accumulator 30.

The electronics module 32 may detect the position or deflection of the diaphragm using diaphragm sensor data from a diaphragm sensor device configured to determine the position (i.e., the amount of deflection) of the diaphragm within the accumulator, such as any of the diaphragm sensors 302, 402, 502, 602 and/or 702 described above with reference to FIGS. 3-7.

In block 806, the electronics module 32 may detect the position or deflection of the diaphragm P₂, at a second time, T₂. The second time T₂ may be later than the first time T₁ by a known or measurement time period (i.e., ΔT). The time period may be less than about 5 seconds, such as less than about 1 second, including less than about a half-second, less than about a quarter second, less than about one-hundredth of a second, less than about a millisecond, etc. The electronics module 32 may detect the position or deflection of the diaphragm, P₂, using diaphragm sensor data from a diaphragm sensor device configured to determine the position (i.e., the amount of deflection) of the diaphragm within the accumulator, such as any of the diaphragm sensors 302, 402, 502, 602 and/or 702 described above with reference to FIGS. 3-7.

The electronics module 32 may determine the change in position or deflection of the diaphragm (i.e., the difference between P₁ and P₂, or ΔP) over the measurement time period, ΔT. As discussed above, the change in position or deflection of the diaphragm as a function of time may be directly related to the rate at which the infusate is pumped from the accumulator. In some embodiments, the electronics module 32 may determine how much the diaphragm moves (i.e., deflects) over a predetermined time period, ΔT. In other embodiments, the electronics module 32 may regularly or continuously monitor the position or deflection of the diaphragm until the diaphragm moves (i.e., deflects) by a pre-determined amount (i.e., ΔP), and may then determine the amount of time elapsed (i.e., ΔT) during the pre-determined change in diaphragm position. For example, the electronics module 32 may be configured to determine the time it takes for the diaphragm to move between an initial upwardly-deflected position P₁ in which the accumulator 30 is in a filled state, as shown in FIG. 2C, to a second position, P₂, in which the diaphragm 40 is fully deflected downwards as shown in FIG. 2A.

In determination block 808, the processor 43 of the electronics module 32 may determine whether the detected change in position or deflection of the diaphragm over the measurement time period (i.e., ΔP/ΔT) satisfies one or more threshold criteria. The at least one threshold criteria may be related to the flow rate of the infusate during normal operation of the implantable drug delivery device. In other words, the detected change in position or deflection of the diaphragm over the measurement time period (i.e., ΔP/ΔT) may be compared to a stored value corresponding to the expected change in position or deflection of the diaphragm over the same time period for a normally-operating device. The detected ΔP/ΔT may satisfy the one or more threshold criteria when the detected ΔP/ΔT deviates from the expected ΔP/ΔT by less than a predetermined amount (e.g., 0-10%). For example, if the detected ΔP/ΔT is less than a first stored threshold value, this may indicate that there is a blockage or occlusion in the flow path of the implantable drug delivery device, and that the flow rate of the device is abnormal. In another example, if the detected ΔP/ΔT is greater than a second stored threshold value (which may be the same or greater than the first threshold value), this may indicate that there is a leak or other problem in the device.

In some embodiments, the processor 43 of the electronics module may optionally determine a flow rate of the accumulator 30 based on the detected change in position or deflection of the diaphragm over the measurement time period (i.e., ΔP/ΔT). For a fixed volume accumulator, a constant volume of infusate is dispensed each time the diaphragm 40 moves from a fully upwardly-deflected position, as shown in FIG. 2C, to a fully-downwardly deflected position, as shown in FIG. 2A. Thus, the change in position or deflection of the diaphragm, ΔP, may be equivalent to a volume, which may be expressed in mL of infusate, for example. Therefore, the detected ΔP/ΔT may be expressed as a flow rate (e.g., mL/sec.), which may be compared to one or more threshold criteria comprising predetermined flow rate value(s) corresponding to normal and/or abnormal flow rates of the implantable drug delivery device.

In response to determining that the detected change in position or deflection of the diaphragm over the measurement time period (i.e., ΔP/ΔT) does not satisfy one or more threshold conditions (i.e., determination block 808=“No”), the processor 43 of the electronics module 32 may determine that the flow rate of infusate is abnormal in block 810. In some embodiments, the determination of an abnormal flow rate may be the result of an occlusion or leak in the implantable drug delivery device. The processor 43 of the electronics module 32 may provide a notification of the abnormal flow rate in block 814. For example, the processor 43 may send a message to an external device 34, such an external programmer, over a wireless interface indicating that the implantable drug delivery device has an abnormal flow rate. The processor 43 may optionally take other remedial action in response to a determination of an abnormal flow rate, such as adjusting the cycling rate of accumulator and/or shutting down the system.

In response to determining that the detected change in position or deflection of the diaphragm over the measurement time period (i.e., ΔP/ΔT) satisfies the one or more threshold conditions (i.e., determination block 808=“Yes”), the processor 43 of the electronics module 32 may determine that the flow rate of infusate is normal in block 810.

In an alternative embodiment, the processor 43 within the implantable drug delivery device may be configured with processor-executable instructions to perform the operations of blocks 804 and 806 and communicate the detected diaphragm position and time values to an external device 34. In this embodiment, the processor 47 of the external programmer 34 may receive the detected values from the implantable drug delivery device and determine whether the flow rate of infusate is normal or abnormal based on a determination of whether the detected change in position or deflection of the diaphragm over the measurement time period (i.e., ΔP/ΔT) satisfies one or more threshold conditions.

As previously discussed, the efficacy of a treatment regimen carried out by the implantable drug delivery devices 300, 400, 500, 600, 700 may be influenced by various impediments (e.g., blockages, occlusions, etc.) within a flow path of each of the implantable drug delivery devices 300. However, other factors may additionally or alternatively influence the treatment regimen carried out by the implantable drug delivery devices 300, 400, 500, 600, 700.

For example, the functionality or efficiency of one or more of the components of the implantable drug delivery devices 300, 400, 500, 600, 700 (e.g., the bellows 16, the filter 24, the accumulator 30, the access port 31, the electronics 32, and/or the catheter 36) may change over time. However, a change in functionality or efficiency may not be significant enough to immediately influence the flow rate and/or the efficacy of the treatment regimen perceived by the patient. Therefore, it may be beneficial to directly or indirectly monitor parameters associated with the functionality of one or more of the components of the implantable drug delivery device 300.

In addition, treatment regimens may include a predetermined dose administration schedule that is defined by a clinician. An amount of infusate prescribed for use during the treatment regimen may be the same amount of infusate used during the treatment regimen. In some embodiments and treatments, a treatment regimen may allow for a predetermined number of patient-initiated doses. Thus, the amount of infusate administered during the treatment regimen may change depending upon whether the patient chooses to use one or more of the allowed number of patient-initiated doses during the treatment regimen. This may result in varying lengths of time that the overall treatment regimen may be performed before the implantable drug delivery device 300 is refilled. Therefore, it may be beneficial to determine the actual infusate flow rate or the amount of infusate administered to a patient within a given time period in order to determine whether the prescribed treatment is being effectuated and/or whether the pump is operating normally.

Various embodiments may determine the flow rate of infusate or the amount of infusate administered to a patient within a given time period based on a select flow rate characteristic, such as a number of accumulator diaphragm displacement cycles and/or a duration (i.e., an amount of time) in which accumulator diaphragm displacement cycles are counted. For example, after a designated number of accumulator diaphragm displacement cycles, a flow rate may be calculated using the time it took to complete those cycles. Alternatively, after a designated period (e.g., a number of seconds, minutes, hours, days, etc. as may be determined by a count-down or count-up timer), a flow rate may be calculated using the number of accumulator diaphragm displacement cycles counted during the designated period. The infusate flow rate may be used to determine various factors, such as an efficacy of a treatment regimen, or detect a change or rate of change in a functionality of the implantable drug delivery device or components thereof (e.g., detect wear out or failure of a component, blockage of a catheter, etc.).

The flow rate characteristic used to calculate the flow rate may be a predetermined value, programmed by the manufacturer, a medical provider, caregiver, patient, or other entity. Alternatively, or additionally, the flow rate characteristic may be set or reset, as an input, during the initiation of the infusate flow rate measurement procedure. As a further alternative, the flow rate characteristic may be determined randomly or using another basis established prior to or during initiation of the infusate flow rate measurement procedure.

The number of performances and/or the time interval between flow rate determinations may vary. The flow rate measurements may be performed by the processor more or less frequently over a time interval associated with one treatment regimen, a plurality of sequential treatment regimens, and/or a lifetime of the implantable drug delivery device. In some embodiments, the flow rate determinations may be performed by the processor more frequently immediately following filling or refilling of the bellows in order to determine an initial maximum flow of infusate for the current treatment regimen. Alternatively, the flow rate determinations may be performed by the processor less frequently following filling or refilling of the bellows 16.

In some embodiments, the processor may perform flow rate determinations more frequently toward the end of the current treatment regimen. For example, in order to prevent an interruption in the delivery of infusate to the patient, it may be desirable to more closely monitor the flow of the infusate to prevent the implantable drug delivery device from emptying the bellows of infusate prior to refilling the bellows 16 with more infusate. In some embodiments, in response to determining that the current flow of the infusate is less than a predetermined threshold but greater than a minimum infusate flow level, the rate at which the flow rate determinations is performed may be increased by the processor to more granularly monitor the consumption of infusate. An alert may be transmitted by the processor to the patient and/or clinician in response to determining that the current flow of the infusate is less than the predetermined threshold value. The alert associated with the infusate level may be provided as an indication or reminder that a refill of infusate is needed within a predetermined amount of time.

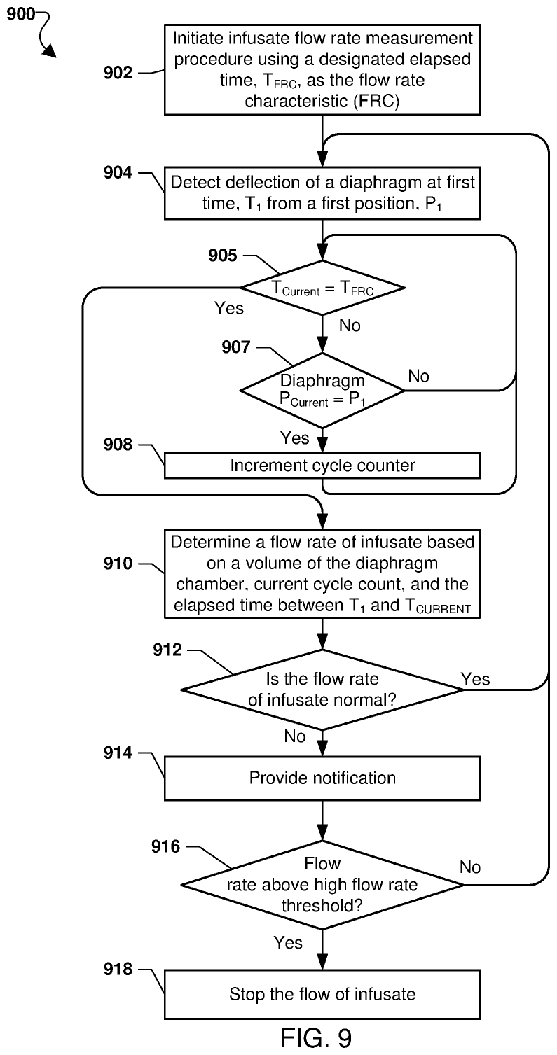

FIG. 9 is a process flow diagram illustrating an embodiment method 900 for a monitoring a flow rate of the infusate from the implantable drug delivery device. With reference to FIGS. 1-9, the method 900 may be implemented by one or more processors of an implantable drug delivery device, a patient programmer, or a combination thereof For example, the method 900 may be implemented by a processor 43 of the electronics module 32 and/or a processor 47 of the external programmer 34.

In block 902, the processor may initiate an infusate flow rate measurement procedure using a designated or predetermined elapsed time, T_{FRC}, as the flow rate characteristic. The processor may initiate the method 900 in various ways, such as continuously, periodically (e.g., repeated at a predetermined interval), episodically (e.g., in response to an internal event or state), or in response to an external input, such from the external programmer (e.g., 34). In some embodiments, the infusate flow rate measurement procedure may be initiated by the processor in response to communication between the external programmer 34 and the electronics modules 32 of the implantable drug delivery device 300. For example, the processor 47 of the external programmer 34 may generate and transmit a message to the electronics module 32 including an instruction to initiate the infusate flow rate measurement procedure. The instruction to initiate the infusate flow rate measurement procedure may be initiated by the external programmer 34 or by the electronics module 32 in response to communications with or inputs to the external programmer 34, such as from a medical provider (e.g., a physician), caregiver, patient, etc.

In some embodiments, the infusate flow rate measurement procedure may be triggered by a predetermined event or operation, such as filling or refilling of the bellows 16 with infusate, receiving a treatment regimen, receiving an indication to modify one or more parameters of the currently implemented treatment regimen, determining otherwise that the flow rate is abnormal, etc.

Additionally or alternatively, the infusate flow rate measurement procedure may be initiated by the processor at periodic intervals during a treatment regimen. The number of times that an infusate flow rate measurement is performed during a treatment regimen and/or a time interval between infusate flow rate measurements may be predetermined or dynamically determined by the processor for a current treatment regimen, for a predetermined number of sequential treatment regimens, and/or for the anticipated life of the implantable drug delivery device. For example, information associated with the number of accumulator diaphragm displacement cycles may be included in the information associated with the current treatment regimen or information associated with the operation of the implantable drug delivery device provided at manufacture, prior to implantation, and/or after implantation may be predefined times or intervals.

In block 904, the processor may detect deflection of the accumulator diaphragm at a first time from a first position within a diaphragm chamber of the accumulator of the implantable drug delivery device. The infusate flow rate measurement procedure may be configured to determine a flow rate of infusate based on a volume of the diaphragm chamber, an accumulator diaphragm displacement cycle count, and an elapsed time. As described herein, the accumulator diaphragm displacement cycle count is the number of cycles the diaphragm goes through when deflecting within the diaphragm chamber, starting at the first position and at the first time, and returning to the first position at the start of a next pumping cycle. The processor may store a value associated with the first time and optionally the first position within the diaphragm chamber. If the first position is always the same (i.e., a predesignated set position, such as at a maximum deflection point), then the processor need not store a value associated with the first position. In some embodiments, the first position may always be the same position (e.g., at a maximum displacement in which the accumulator is either filled with or empty of infusate), which may be predetermined at the time of manufacture or at some other point. Alternatively, the first position may correspond to a starting position of the diaphragm at the initiation of the infusate flow rate measurement (i.e., block 902).

In determination block 905, the processor may determine whether the current time, T_{Current}, equals the designated elapsed time, T_{FRC}. For example, the processor may determine whether a count-down timer set to run for duration T_{FRC} that was started at time T1 has expired. As another example, the processor may determine whether a count up timer started at time T1 has reached T_{FRC}. As a further example, the processor may determine whether an amount of time determined by a clock between time T1 (as recorded) and the current time equals T_{FRC}.

In response to the current time, T_{Current}, not yet equaling the designated elapsed time, T_{FRC} (i.e., determination block 905=“No”), the processor may determine whether the diaphragm has returned to the first position, P₁, (i.e., P_{Current}=P₁) in determination block 907. As discussed above, in some embodiments, the first position may be at either of the opposed walls inside the diaphragm chamber, in which case, a complete cycle of the diaphragm deflection within the diaphragm chamber (i.e., one displacement cycle) starts at the first position, moves to the opposing wall inside the diaphragm chamber, and then returns to the first position. In some embodiments, the first position may be somewhere between the two opposed walls inside the diaphragm chamber, in which case detecting the return of the diaphragm to the first position may include detecting that the diaphragm moved through a complete cycle starting at the first position, moving past the first position, and then returning to the first position. In this way, the diaphragm may need to pass the first position once before returning there in order to complete a full cycle. In such embodiments, the processor may count the number of times the diaphragm passes the first position and then divide by two to determine the number of accumulator diaphragm displacement cycles.

In response to the current diaphragm position, P_{Current}, equaling (being the same as) the starting position, P₁ (i.e., determination block 907=“Yes”), the processor may increment an accumulator diaphragm displacement cycle counter in block 908. The cycle counter may track the number of accumulator diaphragm displacement cycles that the diaphragm has moved through within the diaphragm chamber by incrementing a counter data element in memory each time the diaphragm returns to the start position, P₁. The cycle counter may be reset upon the start of the infusate flow rate measurement procedure (i.e., block 902), thus starting at zero and incrementing from there with each cycle. Alternatively, the cycle counter may be a continuous counter, which may track every accumulator diaphragm displacement cycle since the manufacturer assembled the implantable drug delivery device. In the case of a continuous counter, the processor may need to note or store a starting number of cycles at the start of the infusate flow rate measurement procedure (i.e., in block 902) in order to later determine how many accumulator diaphragm displacement cycles took place between time T₁ and time T_{FRC}. As a further alternative, more than one cycle counter may be maintained by the processor so all accumulator diaphragm displacement cycles over the lifetime of the pump may be tracked, as well as over shorter periods of usage (e.g., T_{FRC}).

In response to determining that the current diaphragm position, P_{Current}, is not at the starting position, P₁ (i.e., determination block 907=“No”) or after incrementing the cycle counter in block 908, the processor may again determine whether the current time, T_{Current}, equals the designated elapsed time, T_{FRC} in determination block 905.

In response to the current time, T_{Current}, equaling the designated elapsed time T_{FRC} (i.e., determination block 905=“Yes”), the processor may determine a flow rate in block 910. Specifically, the processor may determine the flow rate of infusate based on a volume of the diaphragm chamber D_{V}, a current cycle count CC, and the duration between the first time T₁ and the current time T_{Current}. The processor may use a total volume of the diaphragm chamber for D_{V}, which may be programmed into the memory at the time of manufacturing, multiplied by the current cycle count CC, and divided by the time interval between the first and second times to calculate a flow rate. Alternatively, a value less than the maximum volume, such as a measured value of the average dispensed accumulator volume as determined during testing or calibration (or some other measure) may be used by the processor for calculating the flow rate. The processor may implement any of a variety of calculations for determining the flow rate. In an exemplary embodiment, the following equation may be used to determine the flow rate R_{F} of the diaphragm chamber volume D_{V} over the time period between the first time T₁ and the second time T₂:

$${R}_{F} = \frac{CC ⋆ {D}_{V}}{{T}_{Current} - {T}_{1}}$$

In some embodiments or situations, the processor may also or alternatively determine the total volume of infusate that was administered during the measurement period. Specifically, the total volume V_{F} administered between the first time T₁ and the current time T_{Current} may be calculated as: V_{F}=CC*D_{V}.

In determination block 912, the processor may determine whether the flow rate of infusate (or the total volume of administered infusate) is normal based on a comparison of the determined flow rate to one or more threshold criteria (e.g., flow rate threshold(s) or volume threshold(s)). Various embodiments may determine whether the flow rate is considered “normal” by the processor determining whether the current flow rate of infusate is greater than a predetermined low flow rate threshold but less than a predetermined high flow rate threshold. In some embodiments, the threshold criteria (i.e., the low flow rate threshold or the high flow rate threshold) may be predetermined, such as by a clinician configuring the implantable pump upon setting up a treatment protocol, while in other embodiments the threshold criteria may be dynamically determined by the processor based on parameters associated with one or more of the current treatment regimens, a predetermined number of sequential treatment regimens, and/or an anticipated life of the implantable drug delivery device. The threshold criteria may be static for a single treatment regimen or the threshold criteria may be dynamically determined by the processor in response to determining that the administered infusate volume was abnormal. The threshold criteria may be a single value, a pair of values (e.g., high and low thresholds) or a range of values. For example, a flow rate that is 10-15% above a prescribed flow rate may be considered excessive, which may trigger a cut off of the implantable drug delivery device (i.e., stopping the flow of infusate), and a low flow rate threshold (e.g., 10-15% below a prescribed flow rate) may be set to determine when an insufficient dose of infusate is being (or has been) administered. A low flow rate may indicate that there is a partial or complete obstruction inside the accumulator or other components of the pump due to precipitates, catheter kinking, drug incompatibility with valve seat material, scar tissue buildup around the catheter exit, or other reasons.

In response to determining that the flow rate of infusate is normal (i.e., determination block 912=“Yes”), the processor may return to block 904 to again detect deflection of the diaphragm from the same first position, but at a new first time, which starts a new cycle.

In response to determining that the flow rate (or administered volume) of infusate is not normal (i.e., determination block 912=“No”), the processor may provide a notification in block 914. In some embodiments, the notification of abnormal flow rate or volume provided in block 914, may indicate that the determined flow rate does not match a prescribed amount or volume of anticipated infusate or is below or above the prescribed amount or volume by a determined percentage. A difference between a determined flow rate and a prescribed or design flow rate of infusate may be caused by various factors, such as an intentional override of a prescribed dose by the patient, an additional dose administered to the patient, a change in function of one or more elements of the implantable drug delivery device, etc.

In various embodiments, the notification may be provided to the patient and/or a clinician informing them about the abnormal state of the implantable drug delivery device. Such notifications may include sending a notification to an external programmer (e.g., a patient programmer and/or a clinician programmer). In some embodiments, the implantable drug delivery device may include a haptic feedback device (e.g., a shaker), such that the notification includes generating vibrations capable of being perceived by the patient. In some embodiments, the processor may alternatively, or additionally, send a message from the implantable drug delivery device to an external device via a wireless communication link. In this way, the processor may use a transceiver included in the implantable drug delivery device to communicate messages.

In determination block 916, the processor may determine whether the flow rate is above a high flow rate threshold. When infusate is delivered at a rate that is significantly above the prescribed rate, such a condition may lead to overdosing. Thus, in response to the processor determining that the flow rate is above a high flow rate threshold (i.e., determination block 916=“Yes”), the processor may stop the flow of infusate in block 918. In contrast to when too much infusate is being delivered (i.e., above the high flow rate threshold), delivery of too little infusate may be remedied by a physician prescribing a supplement, to compensate for medication not being delivered. Thus, when the infusate deliver rate is too low, it may not warrant stopping the flow of infusate.

In response to the processor determining that the flow rate is not above the high flow rate threshold (i.e., determination block 916=“No”), the processor may return to block 904 to again detect deflection of the diaphragm from the same first position, but at a new first time, which starts a new cycle.

The infusate flow rate measurement procedure in accordance with the method 900 may be interrupted at any time, through input from the electronics module 32 and/or a processor 47 of the external programmer 34. Such an interruption will immediately cause the process to proceed to block 910 to determine a flow rate of infusate based on a volume of the diaphragm chamber, current cycle count, and the elapsed time between T₁ and T_{CURRENT}.

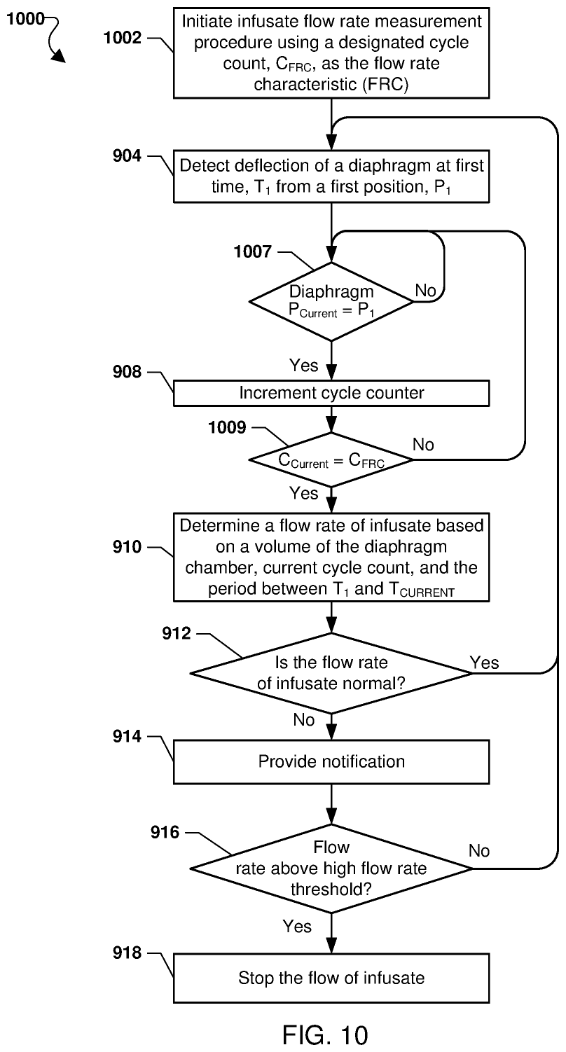

FIG. 10 is a process flow diagram illustrating an embodiment method 1000 for a monitoring a flow rate of the infusate from the implantable drug delivery device. With reference to FIGS. 1-10, the method 1000 may be implemented by one or more processors of an implantable drug delivery device, a patient programmer, or a combination thereof. For example, the method 1000 may be implemented by a processor 43 of the electronics module 32 and/or a processor 47 of the external programmer 34.

In the method 1000, the processor may perform the operations of blocks 904, 908 910, 914, and 918, as well as determination blocks 912 and 916, of the method 900 as described above.

In block 1002, the processor may initiate an infusate flow rate measurement procedure using a designated cycle count, C_{FRC}, as the flow rate characteristic. The processor may initiate the operations of the method 1000 in any of the ways described in block 902 of the method 900.

In determination block 1007, following the operations of block 904, the processor may determine whether the diaphragm has returned to the first position, P₁. (i.e., P_{Current}=P₁). The determination as to whether the diaphragm has returned to the first position, P₁, may be similar to determination block 907 described above for the method 900.

In response to the current diaphragm position, P_{Current}, equaling the starting position, P₁ (determination block 1007=“Yes”), the processor may increment a cycle counter in block 908. In response to the current diaphragm position, P_{Current}, not equaling the starting position, P₁ (determination block 1007=“No”) the processor may again determine whether the diaphragm has returned to the first position, P₁ (i.e., P_{Current}=P₁) in determination block 1007 and continue that cycle until the diaphragm reaches the first position, P₁. Optionally, the processor may have a time-out clock that stops the process after a predetermined time in the event the diaphragm does not return to the first position, P₁ in a timely manner, such as if the diaphragm is stuck, output of the pump is blocked, the pump is empty of infusate, or if the sensor detecting a position of the diaphragm is not registering.

In determination block 1009, following the operations of block 908 (i.e., incrementing the cycle counter), the processor may determine whether the current cycle count, C_{Current}, equals the designated cycle count, CFR. In response to determining that the current cycle count, C_{Current}, does not equal the designated cycle count, C_{FRC}, (i.e., determination block 1009=“No”), the processor may once again determine whether the diaphragm has returned to the first position, P₁ (i.e., P_{Current}=P₁) as part of the next cycle in determination block 1007.

In response to determining that the current cycle count, C_{Current}, equals the designated cycle count, C_{FRC}, (i.e., determination block 1009=“Yes”), the processor may determine a flow rate in block 910 based on the designated cycle count, C_{FRC}, the accumulator volume, and the time required to reach that count, the different between the current time and the time when initiate the operations of the method 1000.

The infusate flow rate measurement procedure in accordance with the method 1000 may be interrupted at any time, through input from the electronics module 32 and/or a processor 47 of the external programmer 34. Such an interruption will immediately cause the process to proceed to block 910 to determine a flow rate of infusate based on a volume of the diaphragm chamber, current cycle count, and the elapsed time between T₁ and T_{CURRENT}.

The foregoing method descriptions and the process flow diagram are provided merely as illustrative examples and are not intended to require or imply that the blocks of the various aspects must be performed in the order presented. As will be appreciated by one of skill in the art the order of blocks in the foregoing aspects may be performed in any order. Words such as “thereafter,” “then,” “next,” etc. are not intended to limit the order of the blocks; these words are simply used to guide the reader through the description of the methods. Further, references to the diaphragm moving “up,” “down,” “upwardly,” and “downwardly” are merely for relating movements of the diaphragm in the orientation illustrated in the figures, and are not intended to limit the scope of the claims regarding a particular orientation of device or diaphragm with respect to the Earth. Further, any reference to claim elements in the singular, for example, using the articles “a,” “an” or “the” is not to be construed as limiting the element to the singular.

The various illustrative logical blocks, modules, circuits, and algorithm blocks described in connection with the aspects disclosed herein may be implemented as electronic hardware, computer software, or combinations of both. To clearly illustrate this interchangeability of hardware and software, various illustrative components, blocks, modules, circuits, and blocks have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A method for monitoring the flow rate of infusate from an implantable drug delivery device, comprising:
detecting deflection of a diaphragm at a first time from a first position within a diaphragm chamber of an accumulator of the implantable drug delivery device;
incrementing a cycle count in response to the diaphragm returning to the first position within the diaphragm chamber during cyclical deflections within the diaphragm chamber;
determining a flow rate of infusate based on a volume of the diaphragm chamber, the cycle count, and an elapsed time since the first time; and
determining whether the flow rate of infusate is normal based on a comparison of the determined flow rate to at least one threshold flow rate.

2. The method of claim 1, wherein determining the flow rate of infusate is performed in response to the elapsed time corresponding to the completion of one of a designated period or the cycle count corresponding to a designated number of cycles.

3. The method of claim 1, wherein incrementing a cycle count in response to the diaphragm returning to the first position within the diaphragm chamber comprises detecting that the diaphragm moved through a complete displacement cycle starting at the first position, moving past the first position, and then returning to the first position.

4. The method of claim 1, wherein the at least one threshold includes a high flow rate threshold, the method further comprising:
stopping the flow of infusate in response to determining that the flow rate of the implantable drug delivery device exceeds the high flow rate threshold.

5. The method of claim 1, further comprising:
providing a notification to at least one of a user of the implantable drug delivery device or a medical professional in response to determining that the flow rate of the implantable drug delivery device is not normal.

6. The method of claim 5, wherein providing a notification comprises sending a message from the implantable drug delivery device to an external device via a wireless communication link.

7. The method of claim 1, wherein detecting the deflection of the diaphragm comprises detecting the change in position using a sensor device of the implantable drug delivery device.

8. The method of claim 7, wherein the sensor device comprises an electronically-based sensor device configured to measure the deflection of the diaphragm within the accumulator.

9. The method of claim 8, wherein the electronically-based sensor device comprises at least one of a strain gauge on a surface of the diaphragm or a capacitive displacement sensor.

10. The method of claim 7, wherein the sensor device comprises a light-based sensor configured to measure the deflection of the diaphragm within the accumulator.

11. The method of claim 7, wherein the sensor device comprises a pressure sensor configured to measure a pressure within the implantable drug delivery device related to the deflection of the diaphragm within the accumulator.

12. The method of claim 7, wherein the sensor device comprises a sonically-based sensor configured to measure the deflection of the diaphragm within the accumulator.

13. An implantable drug delivery device, comprising:
an accumulator comprising a diaphragm chamber and a diaphragm that deflects within the diaphragm chamber to dispense infusate to a patient;
a sensor device configured to measure deflection of the diaphragm within the diaphragm chamber;
a processor coupled to the sensor device and configured with processor-executable instructions to perform operations comprising:
detecting, using data from the sensor device, deflection of the diaphragm at a first time from a first position within the diaphragm chamber;
incrementing a cycle count in response to detecting, using data from the sensor device, that the diaphragm returned to the first position within the diaphragm chamber during cyclical deflections within the diaphragm chamber;
determining a flow rate of infusate based on a volume of the diaphragm chamber, the cycle count, and an elapsed time since the first time; and
determining whether the flow rate of infusate is normal based on a comparison of the determined flow rate to at least one threshold flow rate.

14. The implantable drug delivery device of claim 13, wherein the sensor device comprises at least one of an electronically-based sensor, a light-based sensor, a pressure sensor or a sonically-based sensor.

15. The implantable drug delivery device of claim 13, wherein the sensor device comprises at least one of a strain gauge on a surface of the diaphragm or a capacitive displacement sensor.

16. The implantable drug delivery device of claim 13, wherein the sensor device comprises a light-based sensor configured to detect a change in a light signal related to the deflection of the diaphragm within the accumulator.

17. The implantable drug delivery device of claim 13, wherein the sensor device comprises a pressure sensor configured to detect a change in pressure within a chamber of the implantable drug delivery device related to the deflection of the diaphragm within the accumulator.

18. The implantable drug delivery device of claim 13, wherein the sensor device comprises a sonically-based sensor configured to detect a change in sonic signals related to the deflection of the diaphragm within the accumulator.

19. The implantable drug delivery device of claim 13, further comprising:
a wireless communication transceiver coupled to the processor, wherein the processor is configured with processor-executable instructions to perform operations further comprising:
sending an alert notification to an external device using the wireless communication transceiver in response to determining that the flow rate of the implantable drug delivery device is not normal.

20. An implantable drug delivery device, comprising:
means for detecting deflection of a diaphragm at a first time from a first position within a diaphragm chamber of an accumulator of the implantable drug delivery device;
means for incrementing a cycle count in response to the diaphragm returning to the first position within the diaphragm chamber during cyclical deflections within the diaphragm chamber;
means for determining a flow rate of infusate based on a volume of the diaphragm chamber, the cycle count, and an elapsed time since the first time; and
means for determining whether the flow rate of infusate is normal based on a comparison of the determined flow rate to at least one threshold flow rate.
